(19) United States
(12) Patent Application Publication
(10) Pub. No.: US 2025/0099397 A1
(43) Pub. Date: Mar. 27, 2025

(54) EXTRACELLULAR VESICLES

(71) Applicant: KATHOLIEKE UNIVERSITEIT LEUVEN, Leuven (BE)

(72) Inventors: Guido DAVID, Helecine (BE); Lukas HYKA, Celakovice (CZ); Pascale ZIMMERMANN, Helecine (BE)

(21) Appl. No.: 18/728,230

(22) PCT Filed: Jan. 12, 2023
(86) PCT No.: PCT/EP2023/050644

§ 371 Date: Jul. 11, 2024

(30) Foreign Application Priority Data
Jan. 12, 2022 (EP) 22151092.8

(51) Int. Cl. A61K 9/51; C07K 19/00; C12N 9/22; C12N 15/11

(52) U.S. Cl. CPC A61K 9/5169; C07K 19/00; C12N 9/22; C12N 15/111; C07K 2319/03; C12N 2310/20

(57) ABSTRACT

The invention relates to vesicles comprising: 1) a first fusion protein, the first fusion protein comprising: 1a) a polypeptide capable of binding to a protein on the membrane of a eukaryotic cell, and 2a) a polypeptide capable of binding to syntenin, characterised in that the vesicle further comprises: 2) a second fusion protein, the second fusion protein comprising: 2a) syntenin or a syndecan binding fragment thereof, and 2b) a therapeutic polypeptide, wherein the first fusion protein and the second fusion protein are non-covalenty bound to each other.

### CROSS-REFERENCES TO RELATED APPLICATIONS

This application is a national-stage application under 35 U.S.C. § 371 of International Application No. PCT/EP2023/050644, filed Jan. 12, 2023, which International Applications claims benefit of priority to European Patent Application No. 22151092.8, filed Jan. 12, 2022.

### SEQUENCE LISTING

A computer-readable form (CRF) sequence listing having file name KAT0079PA Substitute Sequence Listing (20,135 bytes), created on Jul. 31, 2024, is incorporated herein by reference.

### TECHNICAL FIELD

The disclosure relates to extracellular vesicles loaded with therapeutic agents, using non-covalent syndecan-syntenin interactions.

### BACKGROUND

Nanoparticulate delivery systems emerge and are utilized to improve the pharmacokinetic and pharmacodynamic profile of therapeutics. It is a problem in the field how to engineer cells to produce nanosized extracellular vesicles carrying and/or protecting high amounts of biological therapeutics and targeting ligands at their surface (enabling vectorization towards specific recipient cells). Moreover the field is awaiting strategies to improve production yields.

Dooley et al. (2021) Mol Ther 29, 1729-1743 and Elsharkasy et al. (2020) Adv Drug Deliv Rev 159, 332-343 review engineered extracellular vesicles with therapeutic agents. Herein the therapeutic agent is always covalently bound to the cell targeting protein. An example hereof is described in Dooley et al. (2021) Mol. Therapy 29(5) 1729-1743, wherein fusion proteins of therapeutic agents with the scaffold protein PTGFRN or BASP1 are sorted into EV. Another example is the pDisplay™ expression system of ThermoFisher, that allows display of proteins on the cell surface. Herein, therapeutic proteins vector are fused at the N-terminus to the murine Ig K-chain leader sequence and at the C-terminus to the platelet derived growth factor receptor (PDGFR) transmembrane domain. This allows display of proteins on the cell surface.

### SUMMARY

The present disclosure provides cells which produce extracellular vesicles (EV) using two types of vectors that permit the enrichment of a targeting signal at the surface of the vesicle, permit efficient encapsulation of a biotherapeutic and permit rewiring of the EV producing cells towards high amounts of engineered exosomes. Herein, the first vector encodes e.g a transmembrane protein fragment of SDC-1 fused to e.g. a nanobody for the targeting to a recipient cell. The second vector encodes a cytosolic syndecan binding peptide (SDCBP1) directly but non-covalently interacting with SDC-1) that supports the sorting inside extracellular vesicles and that is fused to biotherapeutic of interest. This integrated SDC1-SDCBP1 system allows for the coordinated encapsulation of a targeting signal (at the surface of the vesicle) and biotherapeutic (inside the vesicle). Moreover, the overexpression of the SDC1-SDCBP1 selectively boosts the production of engineered extracellular vesicles. The extracellular vesicles of the present disclosure find their cellular target more efficiently (with molecular addresses taking into account target cell heterogeneity). In the compounds and methods of the present disclosure vectorization and the ‘drug’ loading will be coupled (SDC1-CTF-syntenin direct interaction). The fusion of the biotherapeutic, prevents degradation in the cytoplasm of the target cells (leading to dose-effect optimization).

The product and methods of the present disclosure, rewiring exosome producing cells at several levels towards therapeutic exosomes, will equally significantly decrease production costs of therapeutics based on the concept of the disclosure.

One embodiment of the disclosure relates to vesicle comprising a fusion protein, the fusion protein comprising 1) a polypeptide capable of binding to a membrane protein of a mammalian cell and 2) and a polypeptide comprising the cytoplasmic domain of syndecan, characterised in that the vesicle further comprises a further fusion protein comprising 1) syntenin or a syndecan binding fragment thereof and 2) a therapeutic polypeptide.

Herein the polypeptide capable of binding to a membrane protein of a mammalian cell is typically at the N terminal side of the fusion protein, and the polypeptide comprising the cytoplasmic domain of syndecan is typically at the c terminal side of the fusion protein. Herein the syntenin is typically at the N terminal side at the protein and a the therapeutic polypeptide is the c terminal side of the fusion protein.

Examples of a polypeptide capable of binding to a membrane protein of a mammalian cell is a ligand binding domain of a receptor protein, a ligand of a receptor, an antibody or a fragment of an antibody, or a nanobody.

Herein fusion proteins with single chain antibody fragments are envisaged such as an scFv. Other examples are fusion protein with the light chain of an Ab, or fusion proteins with the heavy chain of an Ab, or a fragment thereof. In alternative embodiments, a viral protein that binds to a receptor on a mammalian cell is used.

Typically, the polypeptide capable of binding to a membrane protein of a mammalian cell is a nanobody.

In specific embodiments, wherein the polypeptide capable of binding to a membrane protein of a mammalian cell is connected to the cytoplasmic domain of syndecan via a protease resistant polypeptide.

In specific embodiments, the polypeptide capable of binding to a membrane protein of a mammalian cell is connected to the cytoplasmic domain of syndecan via the Juxta membrane domain of CD4 with sequence VKVLPTWSTXVQPMA, wherein X is P or R [SEQ ID NO:14].

In addition, the fusion protein comprising 1) the polypeptide capable of binding to a membrane protein of a mammalian cell and 2) and comprising the polypeptide comprising the cytoplasmic domain of syndecan, can further comprises a signal protein for translocation and secretion of the fusion protein.

This signal peptide may be the wild type signal of the polypeptide capable of binding to a membrane protein of a mammalian cell. Alternatively the signal peptide is from another protein than polypeptide capable of binding to a membrane protein of a mammalian cell. Typically the signal peptide is at the N terminus of the fusion protein, remote from the sequence of its corresponding mature protein.

An example of such fusion protein has the following structure:

```
MRRAALWLWLCALALSLQPALP-EQKLISEEDL-nanobody sequence- VKVLPTWSTXVQPMA -
Signal peptide myc epitope Juxta domain CD4
syndecan SEQ ID NO: 13 SEQ ID NO: 14
SEQ ID NO: 12
- VLGGVIAGGL VGLIFAVCLV GFMLYRMKKK DEGSYSLEEP KQANGGAYQK PTKQEEFYA
  Cytoplasmic domain syndecan
  SEQ ID NO: 15
Herein in SEQ ID NO: 14 X is P or R
```

In an embodiments of the vesicle the membrane protein of a mammalian cell is syndecan or glypican. In the case of a of syndecan as polypeptide capable of binding to a membrane protein of a mammalian cell, this polypeptide is not directed to the signal peptide and/or the cytoplasmic domain of syndecan.

In typical embodiments the therapeutic polypeptide is a complex of a Cas9 protein and a guide RNA. For example a guide RNA targeting the mutation of an oncogene such as RAS (e.g. G12D or G12V mutation).

In other embodiments the therapeutic polypeptide is selected from the group consisting of a transcription factor a small GTPase regulator protein, a regulator of a kinase, a regulator of a phosphatase, a regulator of a lipase, a kinase, a phosphatase, a lipase.

A second embodiment of the disclosure relates to a kit of eukaryotic expression vectors comprising:
a first vector comprising a nucleotide sequence encoding a fusion protein, the fusion protein comprising 1) a polypeptide capable of binding to a membrane protein of a mammalian cell and 2) and a polypeptide comprising the cytoplasmic domain of syndecan,
a second vector comprising a nucleotide sequence encoding a fusion protein comprising 1) syntenin or a syndecan binding fragment thereof and 2) a therapeutic polypeptide.

The various embodiments described above for the first aspects are equally applicable for the vectors of this kit.

In an embodiment of the kit the polypeptide capable of binding to a membrane protein of a mammalian cell is a nanobody, and wherein a therapeutic polypeptide is a complex of a Cas9 protein and a guide RNA

A further embodiment of the disclosure relates to an eukaryotic cell comprising the vectors of the above kit.

Further embodiments relates to vesicles, kits of vectors, cells, or secreted medium or partially purified medium of such cells comprising the extracellular vesicles, for use as a medicament.

The disclosure is further summarised in the following statements:
1. A vesicle comprising:
1) a first fusion protein, the first fusion protein comprising:
1a) a polypeptide capable of binding to a protein on the membrane of a eukaryotic cell, and
2a) a polypeptide capable of binding to syntenin,
characterised in that the vesicle further comprises:
2) a second fusion protein, the second fusion protein comprising:
2a) syntenin or a syndecan binding fragment thereof, and
2b) a therapeutic polypeptide,
wherein the first fusion protein and the second fusion protein are non-covalenty bound to each other.

Herein the polypeptide capable of binding to a protein on the membrane is located N terminally of a eukaryotic cell is located N terminally of the polypeptide capable of binding to syntenin,

Herein, the syntenin or a syndecan binding fragment thereof is located N terminally of the therapeutic polypeptide.

The eukaryotic cell is typically the cell of a vertebrate, more typically of a mammalian cell, more typically of a human cell.

2. The vesicle according to statement 1, wherein the polypeptide capable of binding to syntenin is syndecan, or a syntenin binding fragment of syndecan.

As indicated in the description on syntenin, other proteins can equally bind to syntenin and can generate the non-covalent binding between the first fusion protein and the second fusion protein.

3. The vesicle according to statement 2, wherein the syndecan is human syndecan 1.

The non-covalent binding of the two fusion can also be performed with other human syndecan, or non-human (e.g. mammalian syndecans as long as it binds to syntenin.

4. The vesicle according to statement 2 or 3, wherein the syntenin binding fragment of syndecan comprises the cytoplasmatic domain of syndecan.

5. The vesicle according to statement 4, wherein the cytoplasmatic domain of syndecan is the polypeptide with SEQ ID NO: 5

6. The vesicle according to any one of statements 2 to 5, wherein the syntenin binding fragment of syndecan further comprises a transmembrane domain sequence, typically a protease resistant transmembrane domain sequence.

7. The vesicle according to statement 6, wherein the transmembrane domain sequence is the transmembrane domain of syndecan.

8. The vesicle according to statement 6, wherein the transmembrane domain sequence is the transmembrane domain of syndecan-1 with SEQ ID NO: 4.

9. The vesicle according to statement 6, wherein the transmembrane domain sequence is the Juxta domain CD4 polypeptide.

10. The vesicle according to statement 9, wherein the Juxta domain CD4 polypeptide has the sequence of SEQ ID NO: 14.

Preferably the Juxta domain CD4 sequence has the sequence of SEQ ID: NO 14 wherein X is P, since it has been shown that this significantly reduces proteolytic cleavage.

11. The vesicle according to any one of statements 1 to 10, wherein the first fusion protein further comprises signal protein for translocation and secretion of the fusion protein.

This signal peptide is typically at the N-terminus of the first fusion protein.

First and or second fusion protein may further contain detectable tags, such as epitope sequence, such as a Myc epitope, an HA tag, or the like.

12. The vesicle according to statement 11, wherein the signal peptide is the signal peptide of syndecan.

13. The vesicle according to statement 11 or 13, wherein the signal peptide is the signal peptide of syndecan with the sequence of SEQ ID NO:2.

Thus in a first fusion protein, when all above features are present the polypeptides are arranged, from N terminal side to C terminal side, as follows: Signal peptide—polypeptide binding to protein on membrane, such as an antibody—transmembrane domain sequence syntenic binding polypeptide such as syndecan and syntenin binding fragments thereof.

This allows a configuration wherein the e.g. antibody is on the outside of the vesicle and the syntenin binding polypeptide is in the inside of the vesicle.

14. The vesicle according to any one of statements 1 to 13, wherein the syndecan binding fragment of syntenin comprises the N-terminal domain, and the PDZ1-PDZ2 tandem domain of syntenin.

15. The vesicle according to any one of statements 1 to 14, wherein the syndecan binding fragment of syntenin further comprises the c terminal domain of syntenin.

16. The vesicle according to any one of statements 1 to 15, wherein syntenin is human synthenin-1 or human syntenin-2.

17. The vesicle according to any one of statements 14 to 16, wherein N terminal domain of syntenin has the sequence of SEQ ID NO: 7.

18. The vesicle according to any one of statements 14 to 17, wherein the PDZ1-PDZ2 tandem domain has the sequence of SEQ ID NO:8.

19. The vesicle according to any one of statements 15 to 18, wherein c terminal domain of syntenin has the sequence of SEQ ID NO:11.

20. The vesicle according to any one of statements 1 to 19, wherein the protein on the membrane is ais an integral membrane protein or a peripheral membrane protein.

21. The vesicle according to any one of statements 1 to 20, wherein the polypeptide capable of binding to a protein on the membrane of a eukaryotic cell is a ligand binding domain of a receptor protein, a ligand of a receptor, an antibody or an antigen binding fragment of an antibody, or a nanobody.

22. The vesicle according to any one of statements 1 to 21, wherein the polypeptide capable of binding to a membrane protein of a mammalian cell is a nanobody.

23. The vesicle according to any of statements 1 to 22, wherein the protein on the membrane is syndecan or glypican.

24. The vesicle according to any one of statements 1 to 23, wherein the therapeutic polypeptide is selected from the group consisting of a transcription factor a small GTPase regulator protein, a regulator of a kinase, a regulator of a phosphatase, a regulator of a lipase, a kinase, a phosphatase, a lipase.

25. The vesicle according to any one of statements 1 to 23, wherein the therapeutic polypeptide is a complex of a Cas9 protein and a guide RNA.

26. The vesicle according to statement 25, wherein the guide RNA targets the mutation of an oncogene, such as RAS.

27. A kit of eukaryotic expression vectors comprising:
a first vector comprising a nucleotide sequence encoding a first fusion protein as defined in any one of statements 1-26, and
a second vector comprising a nucleotide sequence encoding a second fusion protein as defined in any one of statements 1-26.

28. The kit according to statement 27, wherein in the first fusion protein the polypeptide capable of binding to a membrane protein of a mammalian cell is a nanobody, and wherein in the second fusion protein the therapeutic polypeptide is a complex of a Cas9 protein and a guide RNA.

29. A eukaryotic cell comprising the vectors of the kit according to statement 27 or 28.

30. The eukaryotic cell according to statement 29, which is stably transfected with the vectors of the kit of statement 26 or 27.

31. The vesicle according to any of statements 1 to 26, or the kit according to statement 27 or 28, or the cell according to statement 29 or 30, or the secreted medium, or partially purified medium of said cells comprising extracellular vesicles, for use as a medicament.

### DETAILED DESCRIPTION

Figure legends

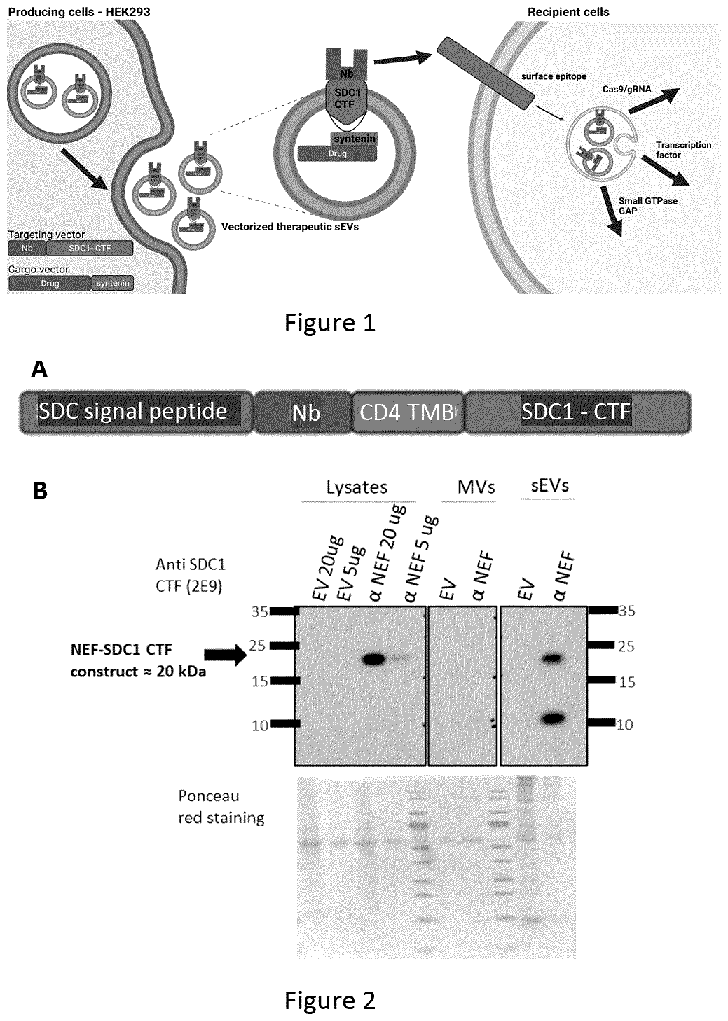

FIG. 1: An exemplary embodiment of the concept of the disclosure.

FIG. 2: SDC1-CTF fusion supports efficient Nb sorting to sEVs.

A. Schematic representation of an expression vector used for nanobody (Nb) sorting at the surface of sEVs. Nbs were N-terminally fused to a SDC1 (syndecan-1) signal peptide and C-terminally fused to CD4 Transmembrane domain (TMD) and SDC1-CTF (syndecan 1 C terminal fragment).

B. Western blot (upper part) and Ponceau of the corresponding membrane (lower part) illustrating the expression of a NEF-Nb construct in lysates, microvesicles (MV) and small extracellular vesicles (sEVs) as indicated.

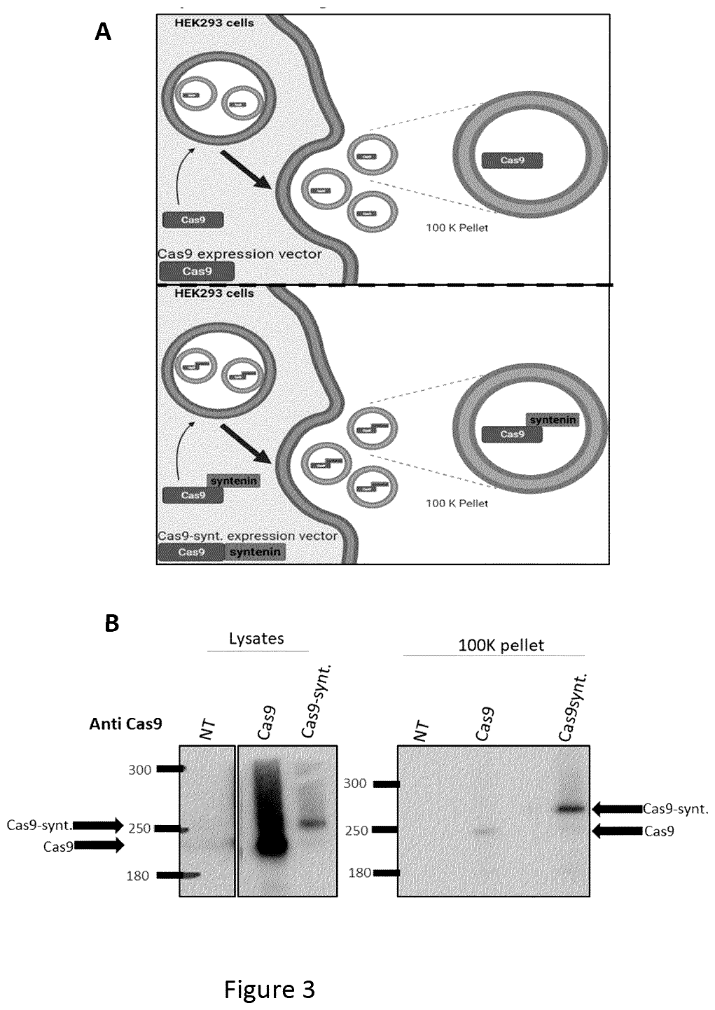

FIG. 3: Syntenin supports efficient sorting of cargo to small extracellular vesicles (sEVs)

A. HEK293 cells transfected with native Cas9 protein (top) or with Cas9-syntenin fusion (bottom).

B. SDS-PAGE and western blot of lysates and pellets.

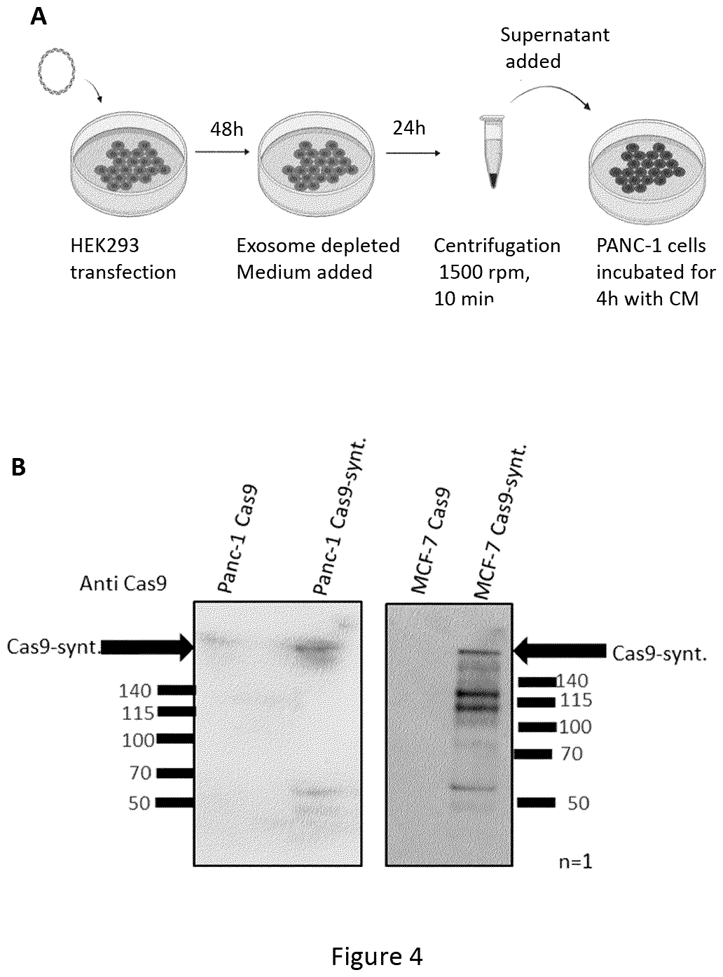

FIG. 4: Syntenin fusion supports efficient delivery of cargo to recipient cells.

A. Design of the experiment to measure HEK293 ev cargo delivery to Panc-1 or MCF-7 cells.

B. SDS PAGE and Western blot of Panc and MCF-7 cells.

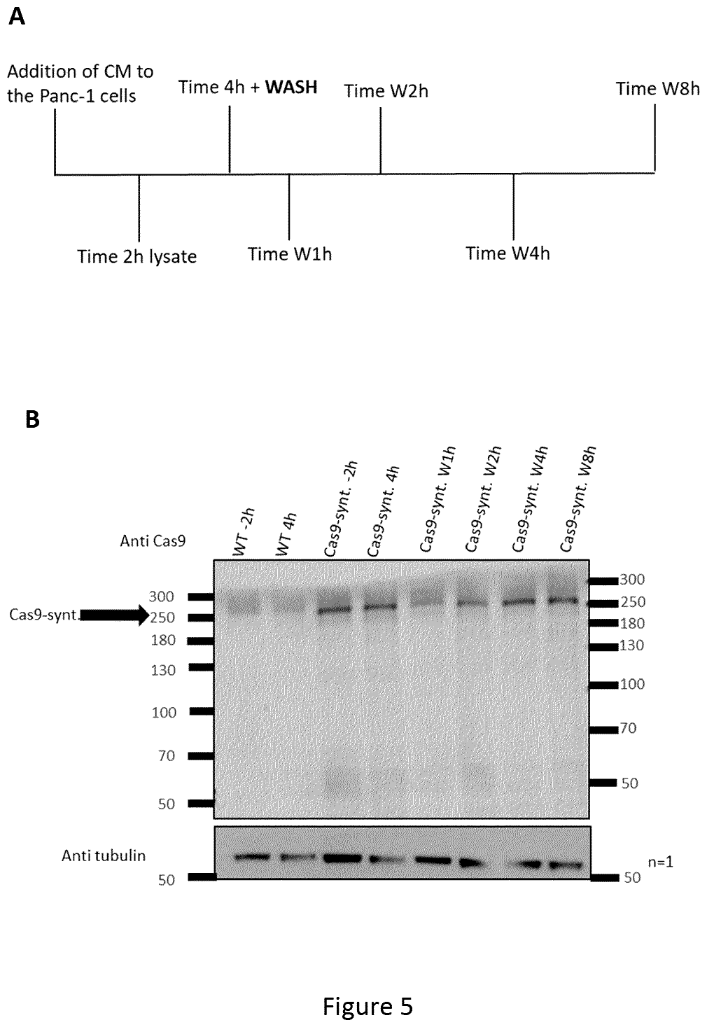

FIG. 5: Syntenin-fused EV cargo remains stable in recipient cells

A. Design of the experiment;

B. SDS PAGE and Western blot of Cas9-synth after different time points.

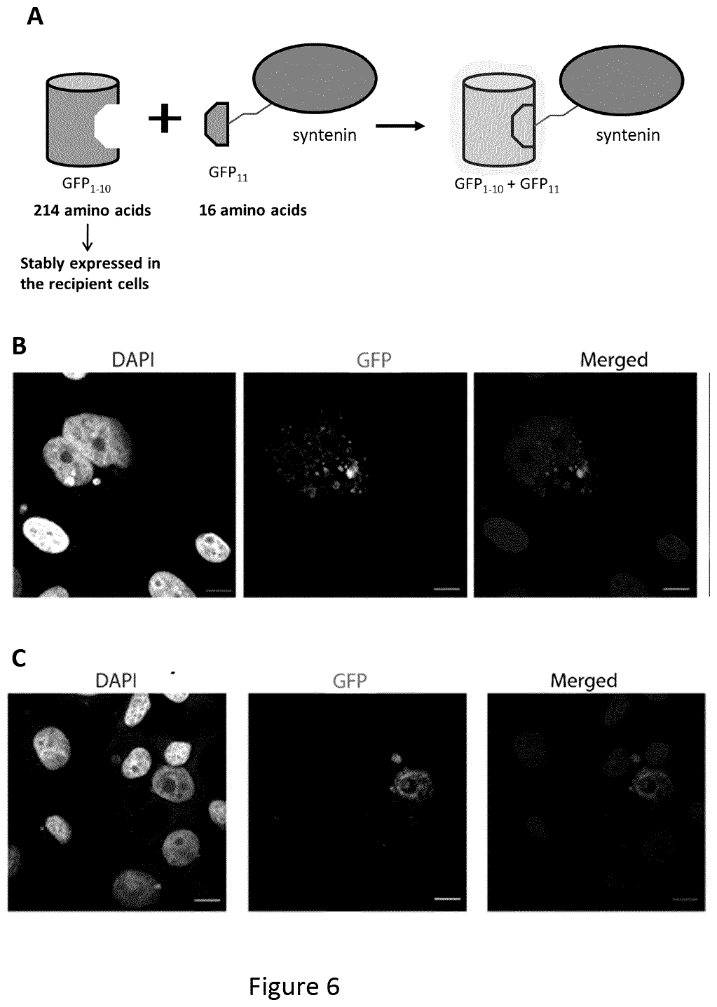

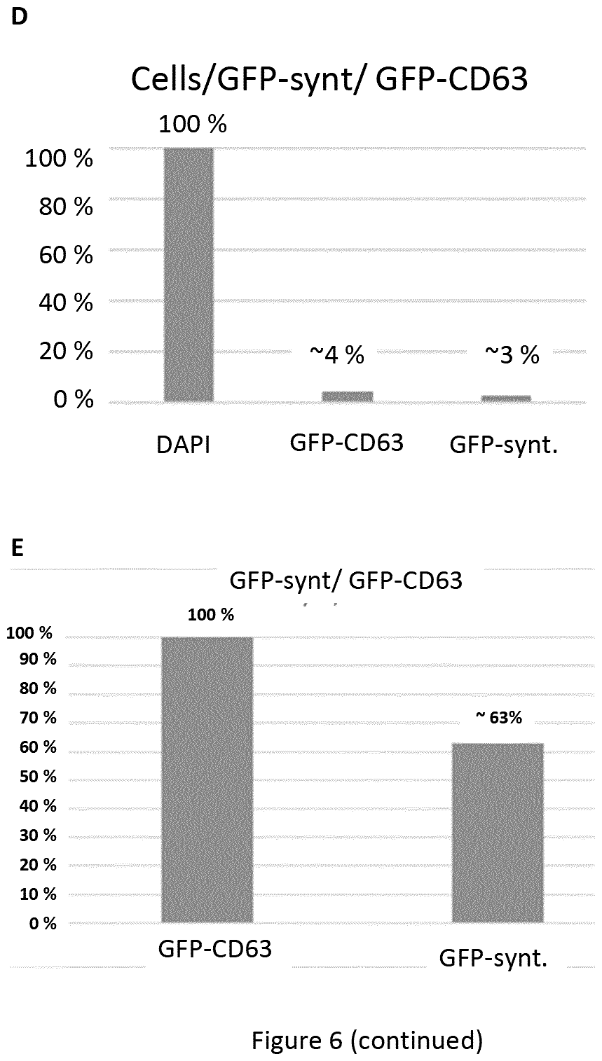

FIG. 6: Syntenin fusion supports endosomal escape in recipient cells

A. Principle of the split Green fluorescent protein (GFP) strategy.

B-C. microscopic images and quantification (D-E) of GFP fusion proteins.

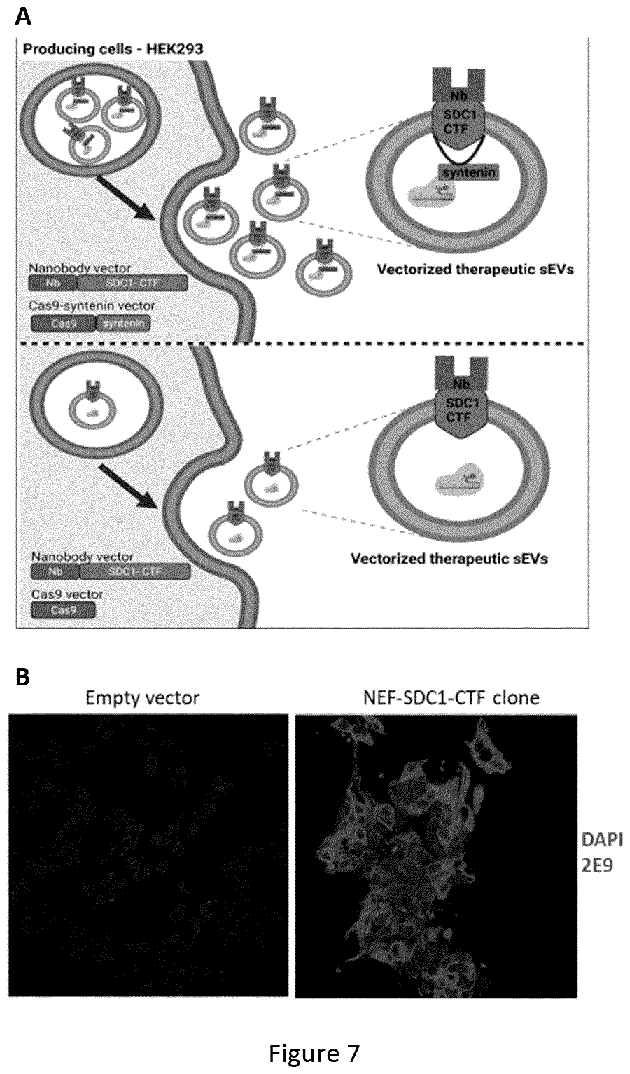

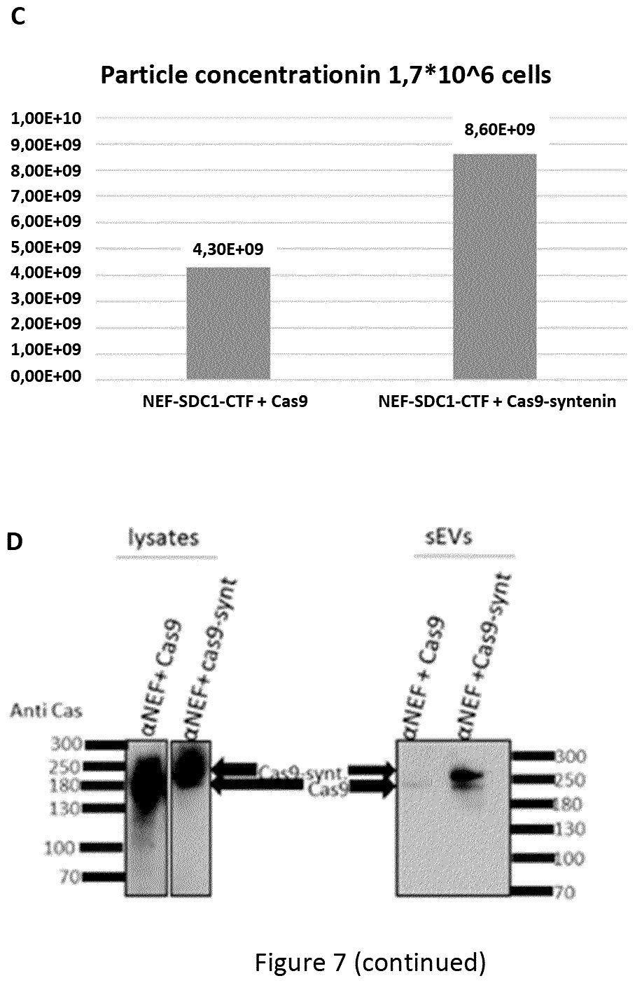

FIG. 7: Expression of Nb-SDC1-CTF plus Cas9-syntenin increases the number of sEVs released and does not impair the sEV sorting of Cas9-syntenin.

A. Illustration of the experimental scheme; B. NEF-SDC1-CTF expression in HEL293 cells; C. sEV particle concentration of NEF-SDC-CTF+Cas9/Cas9-syntenin cells; D. Cas9/Cas9-syntenin sorting to NEF-SDC1-CTF-positive sEVs.

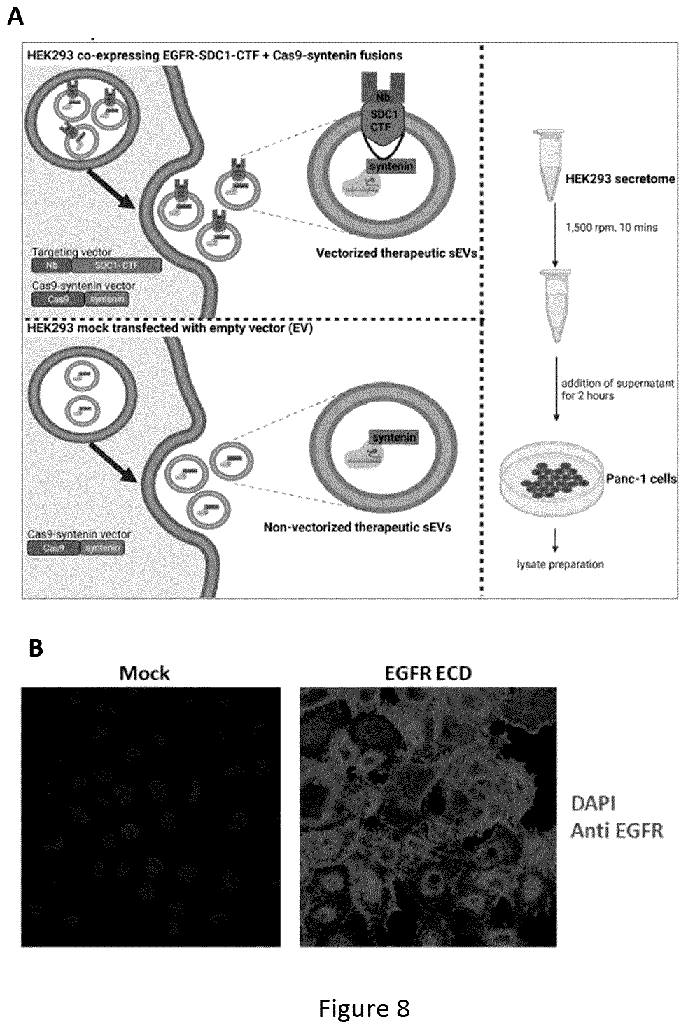

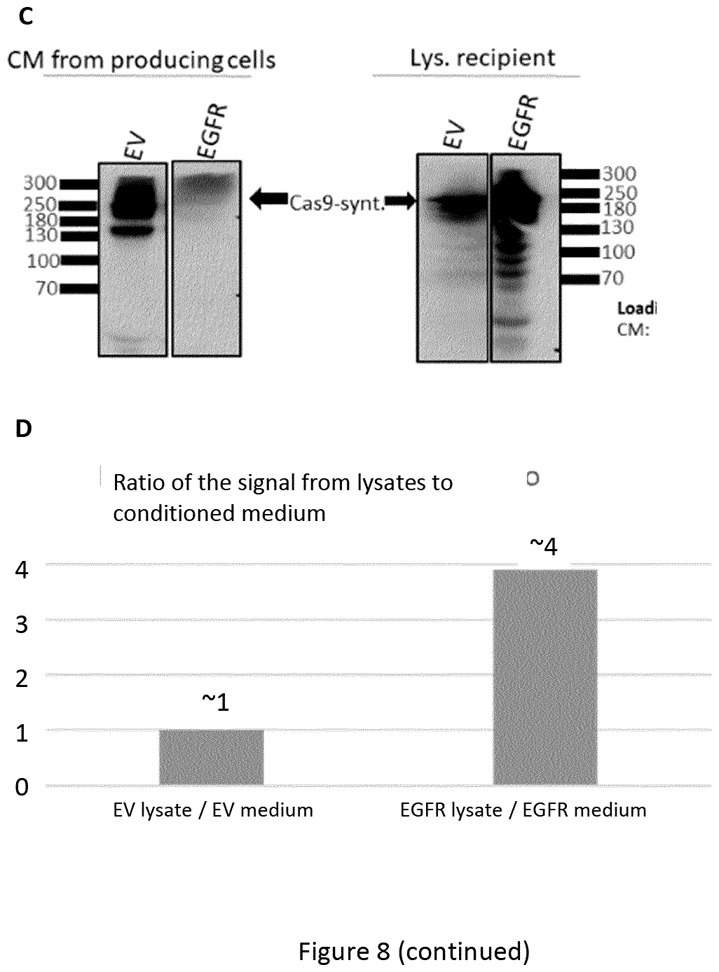

FIG. 8: Vectorization (coating of sEVs with SDC1-CTF-Nb targeting epitopes enriched at the surface of recipient cells) improves the uptake of Cas9-syntenin loaded sEVs by recipient cells.

A. experimental scheme; B. Panc-1 EGFR expression; C. Cas9-syntenin delivery by EGFR positive Evs; D. quantification.

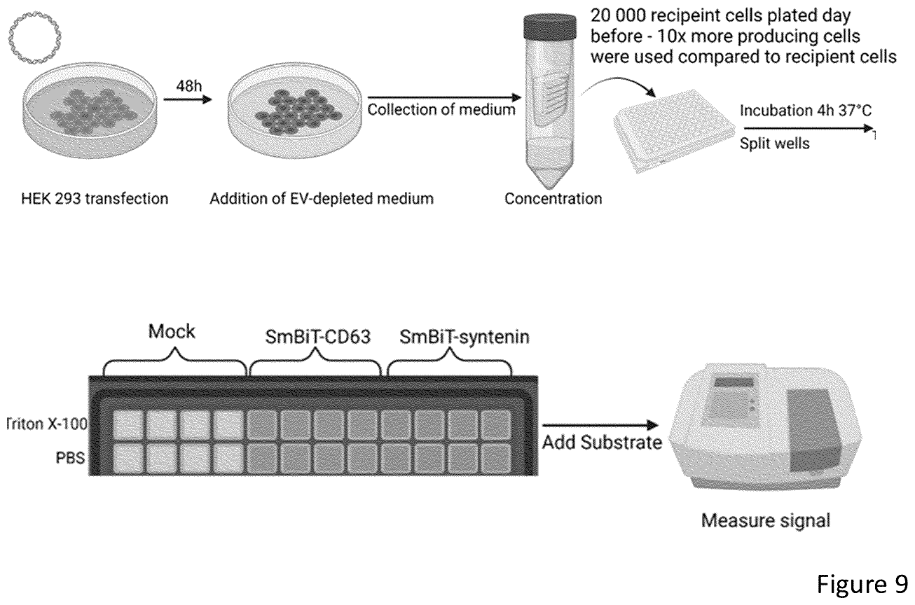

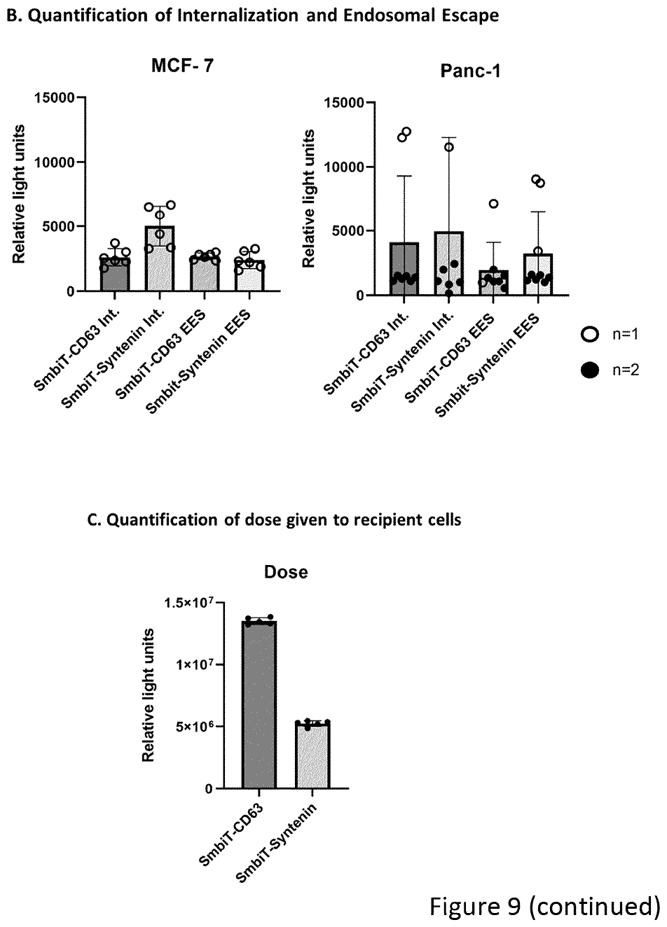

FIG. 9 Validation of endosomal escape using split luciferase.

A. Experimental design. B. Quantification of the internalization (Int.) and endosomal escape (EES) Bar graphs illustrate luciferase signals in relative light units. C. Dose quantification.

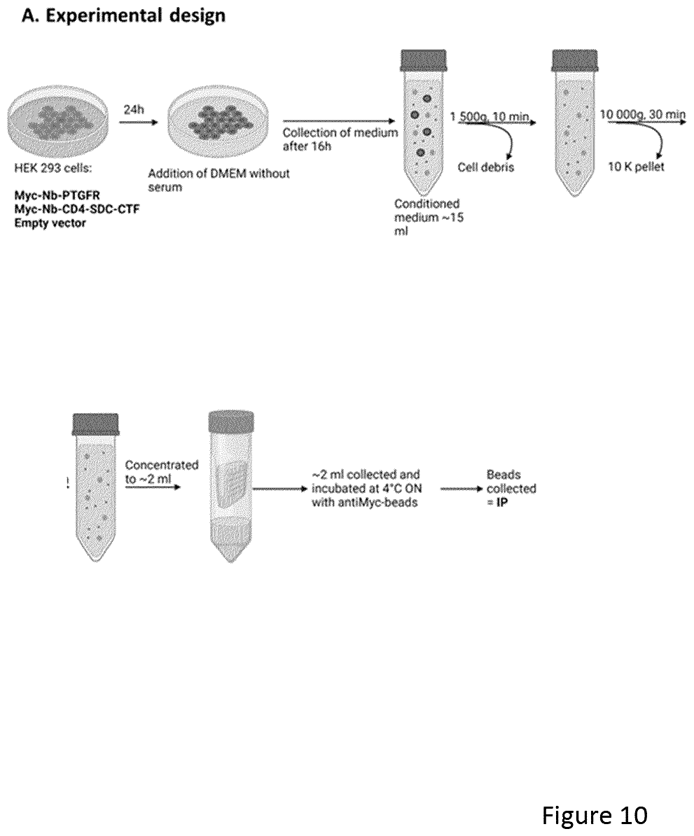

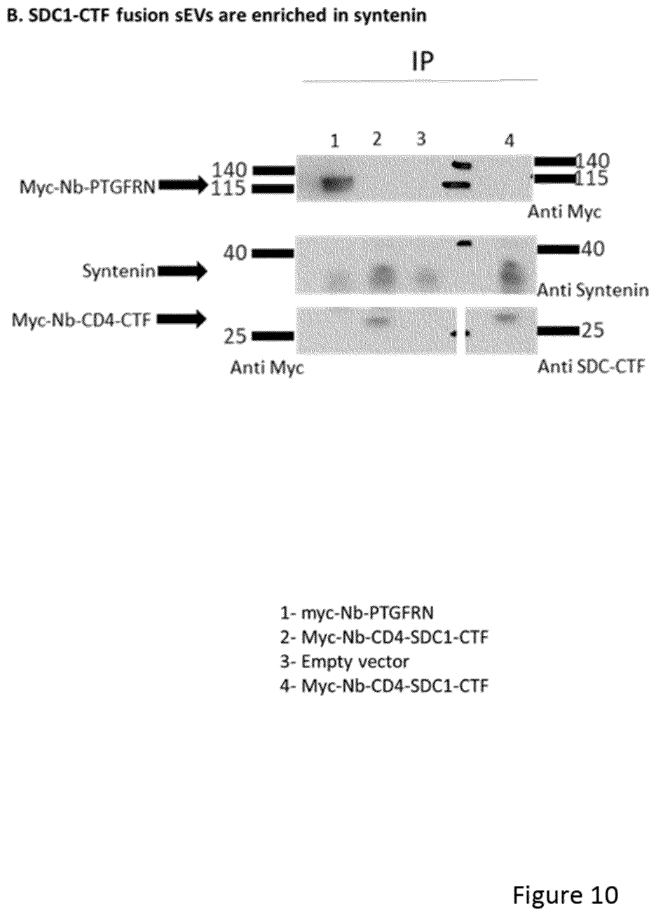

FIG. 10: Verification of syntenin presence in SDC fusion-Nanobody expressing EVs.

A. Experimental design; B. SDC1-CTF fusion sEVs are enriched in syntenin—Western blot analysis of the EV immunoprecipitates (lane 1-4, as indicated).

Abbreviations

### DETAILED DESCRIPTION OF THE DISCLOSURE

EV(s): extracellular vesicle(s)
sEV(s): small extracellular vesicle(s)
SDC: syndecan
SDCBP: syndecan binding protein
CTF: c-terminal fragment

The compounds and methods employ the non-covalent binding of fusion proteins, bases on the interaction of syndecan and syntenin.

Syndecan is a single transmembrane domain protein. The syndecan protein family has four members. Syndecans 1 and 3 and syndecans 2 and 4.

Syndecan has the following protein domains:
a signal sequence;
a extracellular domain
a transmembrane region;
a cytoplasmic domain of about 30 to 35 residue.

These domains are indicated on the human syndecan 1 sequence [SEQ ID NO:1], which serves as a reference for the other syndecans that can be used in the present disclosure.

The equivalent domains in other human syndecans and in syndecans of other sequences can be easily determined by the skilled person in a sequence alignment due to the high sequence similarity.

In the present disclosure a syndecan fragment that binds to syntenin is sufficient to perform the disclosure.

The minimal syntenin binding fragment is typically the C-terminal fragment (CTF) represented by SEQ ID NO: 5.

Mutations and/or even shorter fragments are tolerated as long as the syntenin binding is preserved. This can be determined by in vitro or in vivo binding assays known to the skilled person, such as co-immune precipitations.

In embodiments, the syntenin binding fragment of syndecan further comprises, apart from the CTF, the transmembrane region of syndecan.

In other embodiments the syntenin binding fragment of syndecan further comprises, apart from the CTF and the transmembrane region of syndecan, a extracellular domain.

In other embodiments the entire syndecan sequence is used.

All of the above CTF, transmembrane region+CTF, extracellular domain+ transmembrane region+CTF, or full length syndecan can be used in the first fusion protein of the present disclosure.

Syntenin is a protein that was initially identified as a molecule linking syndecan-mediated signalling to the cytoskeleton.

Furthermore syntenin has been shown to interact with EFNB1, GRIK1, GRIK2, Interleukin 5 receptor alpha subunit, Merlin, RAB5A, SOX4, TRAF6, ULK1 and ERICH2.

Both syntenin-1 and syntenin-2 exist in human.

Syntenins have the following protein domains 1-110

An N terminal domain

A PDZ1-PDZ2 tandem domain

A C terminal domain

In syntenin the N-terminal LYPXL motifs involved in the connection to ESCRT machinery, the PDZ tandem mediates syndecan interaction and connection to lipids (in particular PI4,5P2 and phosphatidic acid), the C-terminus further supports membrane association by electrostatic interactions.

These domains are indicated on the human syntenin-1 sequence [SEQ ID NO: 6], which serves as a reference for the other syntenins that can be used in the present disclosure.

To achieve the advantageous feature of syntenin in the present disclosure (i.e. syndecan binding and prevention of degradation of a fused therapeutic protein in the cytoplasm of the target cells) the minimal syntenin fragment comprises the N terminal domain and the PDZ1-PDZ2 tandem domain.

In other embodiments, the entire syntenin is used.

Mutations and/or even shortened fragments are tolerated as long as the syndecan binding is preserved and escape from protein degradation is preserved. Binding can be determined by in vitro or in vivo binding assays known to the skilled person, such as co-immune precipitations. Protein degradation can be determined in a functional cell assay.

Extracellular vesicles (EVs) are lipid bilayer-delimited particles that are naturally released from almost all types of cell but, unlike a cell, cannot replicate. EVs range in diameter from near the size of the smallest physically possible unilamellar liposome (around 20-30 nanometers) to as large as 10 microns or more, although the vast majority of EVs are smaller than 200 nm. EVs can be divided according to size and synthesis route into Exosomes, microvesicles and apoptotic bodies. They carry a cargo of proteins, nucleic acids, lipids, metabolites, and even organelles from the parent cell.

In the present disclosure the EVs serve as transport vesicle of a fusion protein of a targeting protein and syndecan (or a syntenin binding fragment) and a fusion protein of syntenin (or the N-terminal domain and PDZ domains) and a therapeutic protein. “membrane proteins” are proteins that are attached to, or associated with, the membrane of a cell or an organelle. Membrane proteins are divided into two groups based on the association with the membrane.

“Integral membrane proteins” are permanently embedded within the plasma membrane. They have a range of important functions. Such functions include channeling or transporting molecules across the membrane. Other integral proteins act as cell receptors. Integral membrane proteins can be classified according to their relationship with the bilayer:
Transmembrane proteins span the entire plasma membrane. Transmembrane proteins are found in all types of biological membranes.
Integral monotopic proteins are permanently attached to the membrane from only one side.

“Peripheral membrane proteins” are proteins that are only temporarily associated with the membrane. They can be easily removed, which allows them to be involved in cell signaling. Peripheral proteins can also be attached to integral membrane proteins, or they can stick into a small portion of the lipid bilayer by themselves. Peripheral membrane proteins are often associated with ion channels and transmembrane receptors. Most peripheral membrane proteins are hydrophilic.

An embodiment of the general concept of the disclosure is illustrated in FIG. 1.

The subsequent description describes embodiments of the disclosure, illustrating the concept of the disclosure as more broadly described in the claims.

Herein specific terms are used corresponding to certain features in the claims:
‘address’: protein on the membrane of a cell
‘drug’: therapeutic protein
‘cargo’: second fusion protein
‘cargo vector’: vector encoding second fusion protein
‘targeting vector’: vector encoding first fusion protein

Herein, therapeutic sEVs are produced by HEK293 cells. These cells are genetically modified to secrete sEVs (i) coated with Nanobodies (Nb) recognizing an epitope of a protein at the cell surface of target cells (Targeting vector, encoding first fusion protein, Nb fused to SDC1-CTF) and (ii) loaded with biomolecules targeting disease factors in the recipient cells (Cargo vector, encoding the second fusion protein with a bioactive component fused to syntenin). The SDC1-CTF moiety and the syntenin moiety insure sEV enrichment of the ‘address’ (protein on the membrane of a cell) and the ‘drug’ (therapeutic protein) respectively, and also directly interact, insuring coupling of the ‘address’ and the ‘drug’. Moreover, syntenin overexpression in producing cells rewires them to secrete more syntenic positive sEVs insuring better ‘yields’ of production. Finally, syntenin supports endosomal escape in recipient cells, insuring proper cytosolic delivery of the bioactive component (rather than its degradation upon lysosomal fusion). SDC-1 CTF can be fused to different Nbs, targeting different epitopes at the surface of recipient cells. Syntenin can be used to deliver different types of bioactive components such as: Cas9/gRNA (exemplified here), transcription factors or small GTPase proteins.

The disclosure relates to the design of therapeutic small extracellular vesicles (sEVs) vectorized to display e.g. Nanobodies (‘address’) directed against an epitope of a protein present at the surface of target cells, and enriched in their interior with bioactive components (‘drug’). Synthetic biology is used to engineer the therapeutic sEVs. The Nanobodies (Nb) are concentrated at the surface of sEVs because they are fused to SDC1-CTF. The bioactive component is efficiently loaded into sEVs because it is fused to syntenin. Syntenin also stimulates the production of the sEVs in which it is enriched. The direct molecular interaction between syntenin and SDC1-CTF insure efficient coupling of the ‘address’ and the ‘drug’. Additionally, in the recipient cell, syntenin fusion supports the endosomal escape of the bioactive component or ‘drug’ (insuring escape from lysosomal degradation).

sEVs are typically produced in a HEK293 cell line as these cells are recognized as safe and their products have been extensively studied (Dumont et al. (2016) Crit. Rev. Biotechnol. 36(6), 1110-1122). The HEK293 cells are genetically modified with two types of vectors: the “Targeting vector” (encoding the first fusion protein) and “Cargo vector” (encoding the second fusion protein). In this embodiment, the targeting vector encodes the Nanobocy SDC1-CTF fusion protein displaying the Nb at the surface of sEVs (Roucourt et al. (2015) Cell Res. 25(4), 412-428). The cargo vector encodes the biologically active component fused to syntenin, a polypeptide that is enriched inside of sEVs (Baietti et al. (2012) Nat Cell Biol. 14(7), 677-685). The combination of these fusion proteins results in the coupling of the SDC1-CTF with ‘address’ and syntenin with ‘drug’, both members of the same exosome biogenesis pathway and allowing that the vectorization and cargo loading will happen in the same population of sEVs. Indeed syntenin binds to SDC1-CTF and supports its sorting to sEVs (Baietti et al. cited above). The coupling will increase the yield of therapeutic EV produced. The examples section below further demonstrate that syntenin supports endosomal escape in the recipient cells.

### Example 1. SDC1-CTF Fusion Supports Efficient Nb Sorting to sEVs

1.2 million HEK293 cells stably transfected (clone selected for its homogeneous expression according to immunocytochemistry) with antiNEFnanobody-SDC1-CTF construct (NEF) or empty vector (EV) were plated per 10 cm dish (two dishes per condition). After 48 hours, medium previously depleted of exosomes by ultracentrifugation (18 h, 100,000 g) was added. To concentrate the sEVs, the conditioned medium was collected after 24 hours and centrifuged at 1,500 rpm for 10 minutes, supernatant was collected and centrifuged at 10,000 g for 30 minutes (pellet=microvesicles (MVs)) and the resulting supernatant was centrifuged at 100,000 g for 1.5 h. The pellet was washed by resuspension in 1.4 ml of PBS and centrifuged again at 100,000 g for 1 h (pellet=small extracellular vesicles (sEVs)). Lysates of the producing cells were prepared by scraping the cells and incubating them in RIPA buffer (50 mM Tris-HCl, 150 mM NaCl, 1% NP-40, 0.5% Deoxycholate, 0.1% SDS) for 1 hour followed by centrifugation at 10,000 g for 10 minutes to spin down insoluble material. The lysates and EV preparations were boiled at 95° C. for 10 minutes and loaded on a 4-12% NuPage gradient gels (cat.n. NP0322BOX, ThermoFisher). Proteins were transferred to nitrocellulose membrane (cat.n. 10600018, GE Healthcare) stained for 5 minutes with Ponceau red followed by 1 hour incubation in 5% TPBS/milk and incubated overnight in the presence of mouse 2E9 anti-SDC1 antibody (diluted 1:3.5 in 5% TBS/milk). Primary antibodies were detected by HRP-conjugated (BioRad) secondary antibodies and chemiluminescence (Perkin Elmer). Protein size markers (in kDa) are indicated in red. Lysates corresponding to circa 0.020×10⁶ (20 μg) or 0.005×10⁶ (5 μg) cells and sEVs corresponding to the secretome of circa 5×10⁶ cells were loaded per line. The arrow in FIG. 2B points to full length Nb-SDC1-CTF fusion (approximately 20 kDa). The signal of lower molecular weight corresponds to a cleavage product.

The example tested whether the fusion of Nb to the SDC1-CTF provides selective and efficient sorting of the full-length construct in sEVs. The expression vector contains an open reading frame encoding 4 fused polypeptides generating the synthetic type I transmembrane protein refer to as Nb-SDC1-CTF in FIG. 1. These are N-terminal to C-terminal respectively (i) the signal peptide of SDC1 (cleaved in the endoplasmic reticulum during protein synthesis), (ii) the Nanobody (Nb) (codons optimized for production in mammalian cells), (iii) the CD4 TransMembrane Domain (TMB) to limit membrane proximal cleavage of the fusion construct, and (iv) the SDC1-CTF to optimize sorting into sEVs (FIG. 2A). [CD4 TMD as published in Fitzgerald et al. (2000) J Cell Biol. 148(4), 811-824]. SDC1-CTF was selected since this is enriched in sEVs (Roucourt et al. cited above). EK293 cells stably expressing the Nb-SDC1-CTF fusion protein were prepared and the MVs and sEVs were isolated by differential centrifugation. Lysates of the HEK293 cells were also prepared and both the MV pellets, sEV pellets and lysates were tested for the presence Nb-SDC1-CTF using a monoclonal antibody directed against the cytoplasmic domain of SDC1. A band was detected with molecular size 20 kDa, which is the expected size for the Nb-SDC1-CTF fusion (FIG. 2B). The signal corresponding to the full length of the Nb-SDC1-CTF fusions was detected both in the lysates of the NEF-SDC1-CTF expressing cells as well as in the sEVs fractions. The strong signal for Nb-SDC1-CTF fusion in the sEV fraction indicates efficient sorting. Noteworthy, the lack of the signal in the MVs fraction indicates that the SDC1-CTF fusion directs Nanobodies selectively to sEVs, as aimed for. A cleavage product around 10 kDa was also identified. The level of cleavage varies between experiments and likely due to the processing time. In summary, the experiment demonstrates an efficient coating of sEVs with Nbs.

### Example 2. Syntenin Supports Efficient Sorting of Cargo to Small Extracellular Vesicles (sEVs)

Circa 700,000 HEK293 cells were transfected with 2 ug DNA of Cas9 or Cas9-syntenin expression vector and 6 μl of XtremeGene 9 (cat. n. 6365787001, Merck) resuspended in OptiMEM (cat. n.11058021, ThermoFisher). After 48 hours, medium previously depleted of exosomes by ultracentrifugation (18 h, 100,000 g) was added. To concentrate the sEVs, the conditioned medium was collected after 24 hours and centrifuged at 1,500 rpm for 10 minutes, supernatant was collected and centrifuged at 10,000 g for 30 minutes and the resulting supernatant was centrifuged at 100,000 g for 1.5 h. The pellet was washed by resuspension in 1.4 ml of PBS and centrifuged again at 100,000 g for 1 h (100K pellets). Lysates of the producing cells were prepared by scraping the cells and incubating them in RIPA buffer (50 mM Tris-HCl, 150 mM NaCl, 1% NP-40, 0.5% Deoxycholate, 0.1% SDS) for 1 hour followed by centrifugation at 10,000 g for 10 minutes to spin down insoluble material. The lysates (left panel of FIG. 3B) or sEV preparations (100K pellets, right panel of FIG. 3B), of non-transfected cells (NT), Cas9 transfected cells and Cas9-syntenin transfected cells, as indicated, were boiled at 95° C. for 10 minutes and loaded on the 4-12% NuPage gradient gels (cat.n. NP0322BOX, ThermoFisher). Proteins were transferred to nitrocellulose membrane (cat.n. 10600018, GE Healthcare) followed by 1 hour incubation in 5% TPBS/milk and incubated overnight in the presence of rat anti-Cas9 antibody (cat. n. ab271293, Abcam, diluted 1:5000 in 5% TBS/milk). Primary antibodies were detected by HRP-conjugated (BioRad) secondary antibodies and chemiluminescence (Perkin Elmer). Protein size markers (in kDa) are indicated. Lysates corresponding to circa 0.06×10⁶ and sEVs corresponding to circa 4×10⁶ were loaded per line. Arrows in FIG. 3B point to Cas9 and Cas9-syntenin fusion in lysates and in 100K pellets. Note that in the 100K pellets, the signal for Cas9-syntenin fusion is more intense than that of Cas9 signal, even though Cas9-syntenin fusion signal is weaker in lysates. These data indicate that syntenin supports efficient loading of cargo (Cas9) inside of sEVs.

The example tested whether fusing Cas9 protein to the PDZ protein syntenin can improve Cas9 transfer to sEV, since syntenin is enriched in sEVs and stimulates their formation (Baietti et al. cited above). Native Cas9 or Cas9-syntenin fusion were therefore overexpressed in HEK293 cells and sEVs were isolated by differential centrifugation (FIG. 3A). western blot analysis of the lysates and sEV enriched fractions (100K pellets) was performed with anti-Cas9 antibody. A signal was detected corresponding to the full length Cas9 (approximately 200 kDa) and Cas9-syntenin fusion (approximately 250 kDa). The Cas9-syntenin signal in sEVs is more intense than that of Cas9, while in the lysates it is the Cas9 signal that is more intense than the Cas9-syntenin signal. These data indicate that fusion of cargo (Cas9) to syntenin enhances sorting to sEVs.

### Example 3. Syntenin Fusion Supports Efficient Delivery of Cargo to Recipient Cells

Circa 350,000 HEK293 cells were transfected with 1 ug DNA of Cas9 or Cas9-syntenin expression vector and 3 μl of XtremeGene 9 (cat. n. 6365787001, Merck) resuspended in OptiMEM (cat. n.11058021, ThermoFisher). After 48 hours medium previously depleted of exosomes by ultracentrifugation (18 h, 100,000 g) was added. Conditioned medium was collected after 24 hours and centrifuged at 1,500 rpm for 10 minutes to deplete it from cells and cell debris. The supernatant was added to circa 35,000 MCF-7 or Panc-1 cells. After 4 hours recipient cells were collected by trypsinization, pelleted by centrifugation at 1,000 g for 5 minutes and lysed in Laemmli buffer.

The samples were boiled at 95° C. for 10 minutes and loaded on the 4-12% NuPage gradient gels (cat.n. NP0322BOX, ThermoFisher). Proteins were transferred to nitrocellulose membrane (cat.n. 10600018, GE Healthcare) followed by 1 hour incubation in 5% TPBS/milk and incubated overnight in the presence of rat anti-Cas9 antibody (cat. n. ab271293, Abcam, diluted 1:5000 in 5% TBS/milk). Primary antibodies were detected by HRP-conjugated (BioRad) secondary antibodies and chemiluminescence (Perkin Elmer). Protein size markers (in kDa) are indicated in red. No signal was detected in the Panc-1 and MCF-7 cells incubated with the conditioned medium of Cas9 transfected HEK293 cells. A band corresponding to Cas9-syntenin fusion was detected in Panc-1 and the MCF-7 lysates. The bands migrating at low molecular weight probably correspond to degraded Cas9-syntenin fusion protein. These data indicate that syntenin fusion supports cargo (Cas9) efficient delivery to EV recipient cells.

To test for the delivery of cargo into recipient cells conditioned medium of HEK293 cells transfected with Cas9 or Cas9-syntenin fusion were prepared. The conditioned medium was added to recipient MCF-7 or Panc-1 cells (FIG. 4A). After 4 hours of contact, lysates of the recipient cells were prepared and analyzed by western blot assay for the presence of Cas9 or Cas9-syntenin fusion. Native Cas9 was not detected in the lysates of recipient cell lines. Cas9-syntenin fusion was detected in the lysates for both the Panc-1 and MCF-7 cells (FIG. 4B). These data indicate that syntenin fusion supports efficient delivery of cargo to EV recipient cells.

### Example 4. Syntenin-Fused EV Cargo Remains Stable in Recipient Cells

Circa 700,000 HEK293 cells were transfected with 2 ug DNA of Cas9-syntenin expression vector and 6 μl of XtremeGene 9 (cat. n. 6365787001, Merck) resuspended in OptiMEM (cat. n.11058021, ThermoFisher). After 48 hours, medium previously depleted of exosomes by ultracentrifugation (18 h, 100,000 g) was added. Conditioned medium was collected after 24 hours and centrifuged at 1,500 rpm for 10 minutes to deplete it from cells and cell debris. The supernatant was added to circa 70,000 Panc-1 cells. After 4 hours, the medium was removed, cells were washed with PBS and incubated for indicated periods of time (1 h, 2 h, 4 h, 8 h) in fresh DMEM medium (cat.n. 11965092, ThermoFisher). Hereafter, cells were collected by trypsinization, pelleted by centrifugation at 1,000 g for 5 minutes and lysed in Laemmli buffer. The samples were boiled at 95° C. for 10 minutes and loaded on the 4-12% NuPage electrophoresis gradient gels (cat.n. NP0322BOX, ThermoFisher). Proteins were transferred to nitrocellulose membrane (cat.n. 10600018, GE Healthcare) followed by 1 hour incubation in 5% TPBS/milk and incubated overnight in the presence of rat anti-Cas9 antibody (cat. n. ab271293, Abcam, diluted 1:5000 in 5% TBS/milk) and mouse anti-tubulin (cat.n. 691251, ZioBio, diluted 1:3000 in 5% TBS/milk). Beta-tubulin was used as loading control. Primary antibodies were detected by HRP-conjugated (BioRad) secondary antibodies and chemiluminescence (Perkin Elmer). Protein size markers (in kDa) are indicated. The signal corresponding to the full length Cas9-syntenin fusion (arrowhead, approximately 250 kDa) was detected the cells treated with the Cas9-syntenin conditioned medium. Note that the Cas9-syntenin signal is stable in recipient cells up to 8 hours post incubation with the conditioned medium. These data indicate that syntenin fusion remains stable in recipient cells.

Cas9-syntenin delivery was tested by incubating the Panc-1 cells with the conditioned medium of HEK293 expressing Cas9-syntenin fusion. To determine the stability of Cas9-syntenin fusion recipient cells were washed after 4 hours incubation with the conditioned medium. Recipient cells were then cultured for up to 8 hours in fresh DMEM medium. Cells were collected and lysed at 1 h, 2 h, 4 h, and 8 h after the washing, as indicated (FIG. 5A). The presence of the Cas9-syntenin fusion signal was determined by western blot assay with anti-Cas9 antibody. The band corresponding to Cas9-syntenin fusion stays stable for up to 8 hours after the conditioned medium containing EVs was discarded (FIG. 5B). These data indicate that that Cas9-syntenin fusion remains stable in recipient cells.

### Example 5. Syntenin Fusion Supports Endosomal Escape in Recipient Cells

FIG. 6A shows the principle of the split Green fluorescent protein (GFP) strategy. GFP can be split in two non-fluorescent parts—GFP₁₋₁₀ (214 amino acids) and GFP₁₁ (16 amino acids) (Cabantous & Waldo (2006) Nat. Methods 3(10), 845-854). MCF-7-recipient cells stably expressing the GFP₁₋₁₀ part were prepared. The GFP₁₁ part was fused to syntenin (cytosolic protein) or CD63 (transmembrane protein).

Fluorescence can only be observed upon binding of the GFP₁₋₁₀ with the GFP₁₁ In the present experimental setting, this is only possible when EV content is delivered to the cytosol of recipient cells. GFP₁₁-CD63 (FIG. 6B) or GFP₁₁-syntenin (FIG. 6C) EVs were collected from the conditioned medium of HEK293 cells by ultracentrifugation (100,000 g, 1 h). HEK293 cells were transiently transfected with a GFP₁₁-CD63 (FIG. 6B) or a GFP₁₁-syntenin (FIG. 6C) expression vector. Circa 350,000 cells were transfected with 1 ug of expression vector DNA and 3 μl of XtremeGene 9 (cat. n. 6365787001, Merck) resuspended in OptiMEM (cat. n.11058021, ThermoFisher). The 100,000 g secretome fraction of 350,000 cells was added to 35,000 recipient MCF-7 cells stably expressing GFP₁₋₁₀. After 4 hours, recipient cells were washed, fixed with 4% PFA, stained with DAPI (1 ug in 1 ml PBS) to label nuclei for 10 minutes and mounted in ProLong™ Glass Antifade Mountant (cat. n. P36982, ThermoFisher). The DAPI (left panels) and GFP fluorescence (middle panels) were analyzed by confocal microscopy. Scale bars: 10 um.

FIG. 6D shows a bar graph illustrating the percentage of the GFP positive cells for the GFP₁₁-CD63 (approximately 4%) and GFP₁₁-syntenin (approximately 3%) conditions normalized to DAPI signal. A minimum of 40 cells chosen at random were observed (FIG. 6E). Of note, although GFP₁₁-syntenin is non-transmembrane and can diffuse in the whole recipient cell, the level of GFP₁₁-syntenin-mediated fluorescence reconstitution is almost the as that of GFP₁₁-CD63 that stays localized at membranes. These data indicate that syntenin is efficient at mediating endosomal escape.

GFP split strategy takes advantages of the splitting of GFP in two non-fluorescent parts that become fluorescent only upon reconstitution. This approach was used to measure endosomal escape. HEK293 cells were transfected to express the GFP₁₁ part fused to syntenin or transmembrane protein CD63 (FIG. 6A). GFP₁₁-CD63 positive EVs were used as a control for endosomal escape as endosomal escape of full length GFP-CD63 was already visualized although by an approach not based on the reconstitution of split GFP (Joshi et al. (2020) ACS Nano. 14(4), 4444-4455). Conditioned medium of HEK293 transiently expressing GFP₁₁-CD63 or GFP₁₁-syntenin was collected and EVs were collected by ultracentrifugation. Resuspended EV pellet was added to the MCF-7 cells stably expressing GFP₁₋₁₀. A reconstituted dotty GFP signal was observed in recipient cells upon incubation with GFP₁₁-CD63 positive EVs (FIG. 6B), compatible with GFP reconstitution occurring at membranes. A diffused reconstituted GFP signal was observed in recipient cells upon incubation GFP₁₁-syntenin EVs. The signal was particularly abundant in the nucleus (FIG. 6C). For quantification, 40 cells were selected at random. This shows that endosomal escape occurs in ˜4% of the cells incubated with GFP₁₁-CD63 EVs and ˜3% of the cells incubated with GFP₁₁-syntenin EVs (FIG. 6D). Surprisingly, the reconstituted GFP signal illustrating the endosomal escape of the syntenin fusion protein is almost the same as the reconstituted GFP signal illustrating the endosomal escape of the CD63 control (FIG. 6E). Taken together, these data indicate that fusion to syntenin allows efficient endosomal escape of EV cargo. Moreover, syntenin can also support nuclear enrichment. Backfusion as observed with the split GFP was also obtained using split luciferase, as illustrated below.

### Example 6. Expression of Nb-SDC1-CTF Plus Cas9-Syntenin Increases the Number of sEVs Released and does not Impair the sEV Sorting of Cas9-Syntenin

sEVs were produced from HEK293 cells homogeneously and stably expressing Nb-SDC1-CTF fusion (after clonal selection, see B). These were transiently transfected with an expression vector for Cas9 or for Cas9-syntenin fusion. Circa 1.2×10⁶ cells were transfected by adding 1 ug of corresponding DNA in the presence of 3 μl of XtremeGene 9 (cat. n. 6365787001, Merck) resuspended in OptiMEM (cat. n.11058021, ThermoFisher). After 48 hours post Cas9 or Cas9-syntenin transfection medium previously depleted of exosomes by ultracentrifugation (18 h, 100,000 g) was added. To concentrate the sEVs, the conditioned medium was collected after 24 hours and centrifuged at 1,500 rpm for 10 minutes, supernatant was collected and centrifuged at 10,000 g for 30 minutes and the resulting supernatant was centrifuged at 100,000 g for 1.5 h. The pellet was washed by resuspension in 1.4 ml of PBS and centrifuged again at 100,000 g for 1 h. Lysates of the producing cells were prepared by scraping the cells and incubating them in RIPA buffer (50 mM Tris-HCl, 150 mM NaCl, 1% NP-40, 0.5% Deoxycholate, 0.1% SDS) for 1 hour followed by centrifugation at 10,000 g for 10 minutes to spin down insoluble material. FIG. 7B shows micrographs illustrating the homogeneous expression of the anti-NEF-Nb-SDC-CTF by stably transfected HEK293 cells (after clonal selection, right image). Empty vector HEK293 transfected cells were used as negative controls. Signals were obtained by immunocytochemistry, using 2E9 home-made monoclonal antibody targeting SDC1-CTF. Circa 30,000 cells were plated per a well of 8-well chamber slide (cat. n.154453, ThermoFisher). After 24 hours cells were washed once with PBS and fixed by incubating for 20 minutes with 4% PFA. The cells were stained with mouse 2E9 antibody (diluted 1:1 in solution of 0.3% BSA and saponin 0.05% in PBS) for 1 hour at 4° C. The cells were washed twice with PBS and stained with secondary anti mouse Alexa 555 antibody (cat. n. A-21424, ThermoFisher) diluted 1:1,000 in solution of 0.3% BSA and saponin 0.05% in PBS for 45 minutes in dark at room temperature. The cells were then washed twice with PBS and stained with DAPI (1:1,000) for 10 minutes and mounted in ProLong Glass Antifade Mountant (cat.n. P36982, ThermoFisher). The cells were imaged for the DAPI and Alexa 555 signal by confocal microscopy (FluoView 1000 Olympus). FIG. 7C shows NTA analysis of the sEVs (100.000 g pellet) from the secretome of 1.7×10⁶ cells. The particles were diluted in PBS and their concentration was determined using a ZetaView instrumant (Particle Metrix GmbH, Meerbusch, Germany). To verify that the high amount of Nb-SDC1-CTF protein in donor cells did not impair the presence of Cas9-syntenin in the sEVs pellets Western blot analysis was performed on the 100.000 g pellet (FIG. 7D). The lysates (left panel) or sEV preparations (right panel) were boiled at 95° C. for 10 minutes and loaded on the 4-12% NuPage gradient gels (cat.n. NP0322BOX, ThermoFisher). Proteins were transferred to nitrocellulose membrane (cat.n. 10600018, GE Healthcare) followed by 1 hour incubation in 5% TPBS/milk and incubated overnight in the presence of rat anti-Cas9 antibody (cat. n. ab271293, Abcam, diluted 1:5000 in 5% TBS/milk). Primary antibodies were detected by HRP-conjugated (BioRad) secondary antibodies and chemiluminescence (Perkin Elmer). Protein size markers (in kDa) are indicated in red. Lysates corresponding to circa of 0.012×10⁶ cells (5 ug) and sEVs corresponding to circa 9×10⁶ cells were loaded per line. Arrows point to Cas9 or Cas9-syntenin fusion in lysates and in sEVs pellets. Note, that in the sEVs pellets the signal for Cas9-syntenin fusion is more intense than that of Cas9 signal, and that the Cas9-syntenin fusion signal is weaker than that of Cas9 in lysates. These data indicate that expression of syntenin-fusion protein increases the number of secreted vesicles when co-expressed with a SDC1-CTF fusion. Furthermore, SDC1-CTF fusion does not impair the loading of Cas9-syntenin fusion inside of sEVs.

The effect of the co-expression of both the SDC1-CTF fusion and the syntenin fusion on the number sEVs (FIG. 7A) was tested. A HEK293 clone stably expressing anti-NEF-Nb-SDC1-CTF was selected for high and homogenous expression by immunofluorescence using 2E9 antibody targeting SDC1-CTF (FIG. 7B, right). These cells were transiently transfected with Cas9 or Cas9-syntenin and the concentration of sEVs was analyzed by NTA analysis (FIG. 7C). A 2 fold increase in the number particles in the Cas9-syntenin transfected NEF-SDC1-CTF clones was detected, compared to the Cas9 transfected NEF-SDC1-CTF clones. Such 2-fold increase is remarkable when considering transient transfection experiments (circa 30-50% of cells transfected). Stable and homogeneous expression (100%) of Cas9-syntenin will further raise the number of secreted vesicles. Next, it was investigated whether and how the presence of SDC1-CTF fusion affects the sorting of the Cas9-syntenin fusion to sEVs (FIG. 7D). Western blot analysis of the lysates and sEV fraction (100.000 g pellet) was performed with anti-Cas9 antibody. A signal was detected corresponding to the full length Cas9 (approximately 200 kDa) and Cas9-syntenin fusion (approximately 250 kDa). It was observed that the Cas9-syntenin signal in sEVs is more intense than that of Cas9, while in the lysates it is the Cas9 signal that is more intense than the Cas9-syntenin signal. These data indicate that the expression of the SDC1-CTF fusion and syntenin fusion enhances the secretion of sEVs and do not impair the sorting of Cas9-syntenin to sEVs.

### Example 7 Vectorization (Coating of sEVs with SDC1-CTF-Nb Targeting Epitopes Enriched at the Surface of Recipient Cells) Improves the Uptake of Cas9-Syntenin Loaded sEVs by Recipient Cells

FIG. 8 A illustrates the types of EV producing cells, namely (i) HEK293 cells stably transfected with Nb (anti-EGFR in the present example)-SDC1-CTF fusion and transiently transfected with Cas9-syntenin fusion (upper left) or (ii) HEK293 cells stable transfected with control empty vector (EV, control) and transiently transfected with Cas9-syntenin fusion (lower left). Circa 700,000 HEK293 expressing anti-EGFRNb-SDC1-CTF (clonal population) or control (clonal population) were transfected with 2 ug DNA of the Cas9-syntenin expression vector and 6 μl of XtremeGene 9 (cat. n. 6365787001, Merck) resuspended in OptiMEM (cat. n.11058021, ThermoFisher). After 48 hours post Cas9-syntenin transfection medium previously depleted of exosomes by ultracentrifugation (18 h, 100,000 g) was added. The conditioned medium was collected after 24 hours and centrifuged at 1,500 rpm for 10 minutes to deplete it from cells and cell debris. The supernatant was added to circa 70,000 recipient Panc-1 cells for 2 hours before preparing cell lysate (right). Recipient cells were collected by trypsinization, pelleted by centrifugation at 1,000 g for 5 minutes and resuspended in Laemmli buffer for western blotting (C). To evaluate the presence of sEV loaded with Cas9-syntenin, an aliquot (100 μl) of the conditioned medium was collected and concentrated by 100,000 g centrifugation for 1 h, the resulting pellet was resuspended in Laemmli buffer for western blotting (C). FIG. 8B shows the expression of EGFR in Panc-1 cells as detected by immunocytochemistry using an anti-EGFR antibody against the extracellular domain of EGFR. Circa 30,000 cells were plated per a well of 8-well chamber slide (cat. n.154453, ThermoFisher). After 24 hours cells were washed once with PBS and fixed by incubating for 20 minutes with 4% PFA. The cells were stained with anti EGFR (mouse, sc-120, Santa Cruz) diluted 1:50 in solution of 0.3% BSA and saponin 0.05% in PBS for 1 hour at 4° C. The cells were washed twice with PBS and stained with secondary anti mouse Alexa 555 antibody (cat. n. A-21424, ThermoFisher) diluted 1:1,000 in solution of 0.3% BSA and saponin 0.05% in PBS for 45 minutes in dark at room temperature. The cells were then washed twice with PBS and stained with DAPI (1:1,000) for 10 minutes and mounted in ProLong Glass Antifade Mountant (cat.n. P36982, ThermoFisher). The cells were imagined for the DAPI and Alexa 555 signal. The Panc-1 cells stained only with secondary antibodies are shown as a control (Mock). FIG. 8C shows Western blots illustrating the presence of Cas9-syntenin in the conditioned medium of donor HEK293 cells (left) and in the lysate of Panc-1 recipient after two-hour incubation (right). Samples were boiled at 95° C. for 10 minutes and loaded on the 4-12% NuPage gradient gels (cat.n. NP0322BOX, ThermoFisher). Proteins were transferred to nitrocellulose membrane (cat.n. 10600018, GE Healthcare) followed by 1 hour incubation in 5% TPBS/milk and incubated overnight in the presence of rat anti-Cas9 antibody (cat. n. ab271293, Abcam, diluted 1:5000 in 5% TBS/milk). Primary antibodies were detected by HRP-conjugated (BioRad) secondary antibodies and chemiluminescence (Perkin Elmer). Protein size markers (in kDa) are indicated in red. Conditioned medium corresponding to circa of 0.07×106 producing cells and lysates corresponding to circa 0.07×106 recipient cells were loaded per line. Arrows point to Cas9-syntenin fusion in conditioned medium and in the lysates of recipient cells. FIG. 8D shows quantification of Cas9-syntenin signals in the lysates of recipient cells relative to signals from the conditioned medium. The signals were measured using ImageJ software version 1.53c. These data demonstrate that EGFR-SDC1-CTF improves uptake of Cas9-syntenin EVs by Panc-1 cells by 4-fold.

The present example investigated whether sEVs vectorized with an anti-EGFR-Nb-SDC1-CTF can improve the uptake of sEV Cas9-syntenin by Panc-1 cells. For this, purpose the HEK293 cells stably expressing EGFR-SDC1-CTF fusion, or transfected with empty vector as control, were transfected with a Cas9-syntenin expression vector. The secretome of these cells was added to Panc-1 cells (FIG. 8A). Panc-1 cells were used as they highly express EGFR (FIG. 8B). Western blot analysis was performed to test for the presence of Cas9-syntenin in the conditioned medium and the lysates from recipient cells (FIG. 8C). It was observed that uptake occurs by using both the vectorized (EGFR lane) or non. vectorized (EV lane) extracellular vesicles as Cas9-syntenin was detected in the lysate of recipient cells for both conditions. When comparing the signal from the lysates to the signal of the conditioned medium it can be seen that the uptake of EGFR-SDC1-CTF vectorized vesicles is enhanced compared to the non. vectorized extracellular vesicles (FIG. 8D). These data indicate that vectorization of EVs with anti-EGFR-Nb-SDC1 are outperforming non-vectorized EVs to reach their target.

### Example 8 Validation of Endosomal Escape Using Split Luciferase

The experimental design is depicted in FIG. 9 A—Endosomal escape was measured by split NanoLuc approach. In this approach luciferase is reconstituted when the SmbiT fragment and LgbiT fragment meet. HEK293 cells were transiently transfected with expression vectors for SmbiT-syntenin, SmbiT-CD63 or Mock control. 48 h after transfection, cells were washed and EV-depleted medium was added for 16 h. This HEK293 conditioned medium thus contained vesicles loaded with SmbiT-syntenin, SmbiT-CD63 or no SmbiT fusion. The medium underwent 1500 g centrifugation step for 10 minutes to discard cell debris. Next, the medium was concentrated with Amicon 3K filter and added to recipient cells. The overall percentage of concentrated conditioned medium per well did not exceed 20% and the conditioned medium of 10 HEK293 cell per recipient cell was used. The medium was added to recipient cells stably and homogeneously expressing LgbiT (large part of NanoLuc). MCF-7 cells (breast cancer cell line) and Panc-1 cells (pancreatic cancer cell line) were used as recipient cells. The concentrated conditioned medium was incubated at 37° C. for 4 hours with recipient cells. MCF-7 cells (breast cancer cell line) and Panc-1 cells (pancreatic cancer cell line) were used as recipient cells. After incubation, the medium was removed and the recipient cells were split between two wells after trypsinization. The first well was incubated with 1% Triton X 100 to measure the signal obtained from all internalized vesicles. The second well was kept without Triton to measure only the signal from vesicles that underwent direct fusion with the plasma membrane or more probably endosomal escape (fusion of the vesicle membrane with the membrane of the multivesicular endosome-release of the vesicle intraluminal content in the cytosol). To each well a luciferase substrate Furimazine (final concentration 20 uM per well) was added and the wells were incubated at room temperature on the orbital shaker for 5 minutes to distribute the substrate equally. After 5 minutes the luciferase signal was measured by Victor plate reader (PerkinElmer). The measured Mock signal was averaged and the average Mock signal was substracted from the signal of each individual well for the SmbiT-CD63/syntenin samples. FIG. 9B show Quantification of the internalization (Int.) and endosomal escape (EES). Bar graphs in FIG. 9B illustrate luciferase signals in relative light units with mean values for Panc-1 cells (4119.5 in Int. SmBiT-CD63, 4972.1 for Int. SmbiT-Syntenin, 1971.75 for EES SmbiT-CD63 and 3241.9 for EES SmbiT-syntenin) and for MCF-7 cells (2621.7 in Int. SmBiT-CD63, 5038.7 for Int. SmbiT-Syntenin, 2679.7 for EES SmbiT-CD63 and 2400 for EES SmbiT-syntenin) for the different samples as indicated on the X-axis. Internalization (Int.) bars correspond to the luminescence signal measured in the recipient cells in the presence of detergent (total signal). Endosomal escape (EES) bars correspond to the luminescence signal measured in the recipient cells in the absence of detergent (EVs that underwent EES or possibly fusion with the plasma membrane). Int. signals differ between the two recipient cell types, which may indicate different uptake efficiencies. In MCF-7 cells, SmbiT-CD63 appears to undergo 100% EES while this drops to circa 50% in Panc-1 cells. For SmbiT-Syntenin around 50% EES was measured in MCF-7 cells and circa 65% in Panc-1 cells. In summary, these data show that syntenin-fusion allows efficient endosomal escape, comparable to four membrane span CD63 fusion.

FIG. 9C shows the dose quantification. Total EV doses were also measured before internalization (EVs were permeabilized by 0.1% Triton X 100 and incubated in the presence of excess of LgbiT recombinant protein). Herein the total EV doses signals were circa 3 times higher for SmbiT-CD63 than for SmbiT-syntenin.

These results demonstrate that syntenin-fused cargo transported by EVs escapes degradation in recipient cells.

### Example 9: Verification of Syntenin Presence in SDC Fusion-Nanobody Expressing EVs

The setup of the experiment is depicted in FIG. 10A.HEK293 cells stably expressing Myc-Nb-PTGFRN, Myc-Nb-CD4-SDC1-CTF fusion (these cells secrete EVs with myc epitope on the surface) or empty vector cells (not expressing myc epitope, no specific binding to the anti myc beads) were plated on 10 cm dishes. After 24 hours medium was exchanged for DMEM (in the absence of FBS). After additional 16 hours the medium from each of the 3 conditions was collected and subjected to two rounds of centrifugation (1500 g to remove cell debris and 1000 g to remove large EVs). The medium was concentrated to approximately 2 ml using Amicon 3K filter and the concentrated medium containing secreted EVs was incubated with anti-myc agarose beads (Chromotek, cat.n. yta) at 4° C. overnight. Next day, the beads were pelleted by low speed centrifugation washed 5× times in PBS and subsequently boiled in 5× Laemmli buffer.

Myc-Nb-PTGFRN was detected using anti-myc antibody (upper blot FIG. 10B). Myc-Nb-CD4-SDC1-CTF fusion was detected with anti-myc antibody (bottom left blot FIG. 10B) or anti SDC-CTF antibody (bottom right blot FIG. 10B). Endogenous syntenin was detected using anti syntenin antibody (Kashyap et al. (2021) Sci Rep. 11(1), 4083) (middle blot). Herein, the syntenin signal observed for the Myc-Nb-PTGFRN is similar to the background signal observed for the immunoprecipitate of non-myc-tagged EVs (lane 1 and 3). Syntenin enrichment occurs in Myc-Nb-CD4-SDC1-CTF positive EVs (lane 2 and 4). This further demonstrated that syntenin is enriched in the SDC positive EVs and not in SDC negative EVs. This further illustrates the advantage of using SDC fusions to load surface molecules non-colavently coupled to syntenin with therapeutic proteins.

Sequences in sequence listing

Human syndecan-1 [SEQ ID NO: 1]

| | |
|---|---|
| MRRAALWLWL CALALSLQLA LPQIVATNLP PEDQDGSGDD SDNFSGSGAG | 50 |
| ALQDITLSQQ TPSTWKDTQL LTAIPTSPEP TGLEATAAST STLPAGEGPK | 100 |
| EGEAVVLPEV EPGLTAREQE ATPRPRETTQ LPTTHQASTT TATTAQEPAT | 150 |
| SHPHRDMQPG HHETSTPAGP SQADLHTPHT EDGGPSATER AAEDGASSQL | 200 |
| PAAEGSGEQD FTFETSGENT AVVAVEPDRR NQSPVDQGAT GASQGLLDRK | 250 |
| EVLGGVIAGG LVGLIFAVCL VGFMLYRMKK KDEGSYSLEE PKQANGGAYQ | 300 |
| KPTKQEEFYA | 310 |

1- 17 signal sequence [SEQ ID NO: 2]

| | |
|---|---|
| 1 MRRAALWLWL CALALSL | 17 |

18-251 extracellular domain (ectodomain) [SEQ ID NO: 3]

| | |
|---|---|
| 18 QLA LPQIVATNLP PEDQDGSGDD SDNFSGSGAG | 50 |
| ALQDITLSQQ TPSTWKDTQL LTAIPTSPEP TGLEATAAST STLPAGEGPK | 100 |
| EGEAVVLPEV EPGLTAREQE ATPRPRETTQ LPTTHQASTT TATTAQEPAT | 150 |
| SHPHRDMQPG HHETSTPAGP SQADLHTPHT EDGGPSATER AAEDGASSQL | 200 |
| PAAEGSGEQD FTFETSGENT AVVAVEPDRR NQSPVDQGAT GASQGLLDRK | 250 |
| E | 251 |

252-276 transmembrane region [SEQ ID NO: 4]

| | |
|---|---|
| 252 VLGGVIAGG LVGLIFAVCL VGFMLY | 276 |

277-310 cytoplasmic domain [SEQ ID NO: 5]

| | |
|---|---|
| 277 RMKK KDEGSYSLEE PKQANGGAYQ KPTKQEEFYA | 310 |

Human syntenin-1 sequence [SEQ ID NO: 6]

| | |
|---|---|
| MSLYPSLEDL KVDKVIQAQT AFSANPANPA ILSEASAPIP HDGNLYPRLY | 50 |
| PELSQYMGLS LNEEEIRASV AVVSGAPLQG QLVARPSSIN YMVAPVTGND | 100 |
| VGIRRAEIKQ GIREVILCKD QDGKIGLRLK SIDNGIFVQL VQANSPASLV | 150 |
| GLRFGDQVLQ INGENCAGWS SDKAHKVLKQ AFGEKITMTI RDRPFERTIT | 200 |
| MHKDSTGHVG FIFKNGKITS IVKDSSAARN GLLTEHNICE INGQNVIGLK | 250 |
| DSQIADILST SGTVVTITIM PAFIFEHIIK RMAPSIMKSL MDHTIPEV | 298 |

1-110 N terminal domain [SEQ ID NO: 7]

| | |
|---|---|
| MSLYPSLEDL KVDKVIQAQT AFSANPANPA ILSEASAPIP HDGNLYPRLY | 50 |
| PELSQYMGLS LNEEEIRASV AVVSGAPLQG QLVARPSSIN YMVAPVTGND | 100 |
| VGIRRAEIKQ | |

111-272 PDZ1-PDZ2 tandem domain [SEQ ID NO: 8]

| | |
|---|---|
| 111 GIREVILCKD QDGKIGLRLK SIDNGIFVQL VQANSPASLV | 150 |
| GLRFGDQVLQ INGENCAGWS SDKAHKVLKQ AFGEKITMTI RDRPFERTIT | 200 |
| MHKDSTGHVG FIFKNGKITS IVKDSSAARN GLLTEHNICE INGQNVIGLK | 250 |
| DSQIADILST SGTVVTITIM PA | 272 |

111-198 PDZ1 domain [SEQ ID NO: 9]

| | |
|---|---|
| 111 GIREVILCKD QDGKIGLRLK SIDNGIFVQL VQANSPASLV | 150 |
| GLRFGDQVLQ INGENCAGWS SDKAHKVLKQ AFGEKITMTI RDRPFERT | 198 |

195-272 PDZ2 domain [SEQ ID NO: 10]

| | |
|---|---|
| 195 FERTIT | 200 |
| MHKDSTGHVG FIFKNGKITS IVKDSSAARN GLLTEHNICE INGQNVIGLK | 250 |
| DSQIADILST SGTVVTITIM PA | 272 |

273-298 C terminal domain [SEQ ID NO: 11]

| | |
|---|---|
| 273 FIFEHIIK RMAPSIMKSL MDHTIPEV | 298 |

Signal peptide syndecan [SEQ ID NO: 12]
MRRAALWLWLCALALSLQPALP
myc epitope [SEQ ID NO: 13]
EQKLISEEDL
Juxta domain CD4 [SEQ ID NO: 14]
VKVLPTWSTXVQPMA
X is P or R
Transmembrane domain and cytoplasmic domain syndecan [SEQ ID NO: 15]
VLGGVIAGGL VGLIFAVCLV GFMLYRMKKK DEGSYSLEEP KQANGGAYQK PTKQEEFYA

1. A vesicle comprising:
1) a first fusion protein, the first fusion protein comprising:
1a) a polypeptide capable of binding to a protein on the membrane of a eukaryotic cell, and
2a) a polypeptide capable of binding to syntenin,
characterised in that the vesicle further comprises:
2) a second fusion protein, the second fusion protein comprising:
2a) syntenin or a syndecan binding fragment thereof, and
2b) a therapeutic polypeptide,
wherein the first fusion protein and the second fusion protein are non-covalenty bound to each other.

2. The vesicle according to claim 1, wherein the polypeptide capable of binding to syntenin is syndecan, or a syntenin binding fragment of syndecan.

3. The vesicle according to claim 2, wherein the syndecan is human syndecan 1.

4. The vesicle according to claim 2 or 3, wherein the syntenin binding fragment of syndecan comprises the cytoplasmatic domain of syndecan.

5. The vesicle according to claim 4, wherein the cytoplasmatic domain of syndecan is the polypeptide with SEQ ID NO: 5

6. The vesicle according to any one of claims 2 to 5, wherein the syntenin binding fragment of syndecan further comprises a transmembrane domain sequence, typically a protease resistant transmembrane domain sequence.

7. The vesicle according to claim 6, wherein the transmembrane domain sequence is the transmembrane domain of syndecan.

8. The vesicle according to claim 6, wherein the transmembrane domain sequence is the transmembrane domain of syndecan-1 with SEQ ID NO: 4.

9. The vesicle according to claim 6, wherein the transmembrane domain sequence is the Juxta domain CD4 polypeptide.

10. The vesicle according to claim 9, wherein the Juxta domain CD4 polypeptide has the sequence of SEQ ID NO:14.

11. The vesicle according to any one of claims 1 to 10, wherein the first fusion protein further comprises signal protein for translocation and secretion of the fusion protein.

12. The vesicle according to claim 11, wherein the signal peptide is the signal peptide of syndecan.

13. The vesicle according to claim 11 or 13, wherein the signal peptide is the signal peptide of syndecan with the sequence of SEQ ID NO:2.

14. The vesicle according to any one of claims 1 to 13, wherein the syndecan binding fragment of syntenin comprises the N-terminal domain, and the PDZ1-PDZ2 tandem domain of syntenin.

15. The vesicle according to any one of claims 1 to 14, wherein the syndecan binding fragment of syntenin further comprises the c terminal domain of syntenin.

16. The vesicle according to any one of claims 1 to 15, wherein syntenin is human synthenin-1 or human syntenin-2.

17. The vesicle according to any one of claims 14 to 16, wherein N terminal domain of syntenin has the sequence of SEQ ID NO: 7.

18. The vesicle according to any one of claims 14 to 17, wherein the PDZ1-PDZ2 tandem domain has the sequence of SEQ ID NO:8.

19. The vesicle according to any one of claims 15 to 18, wherein c terminal domain of syntenin has the sequence of SEQ ID NO:11.

20. The vesicle according to any one of claims 1 to 19, wherein the protein on the membrane is ais an integral membrane protein or a peripheral membrane protein.

21. The vesicle according to any one of claims 1 to 20, wherein the polypeptide capable of binding to a protein on the membrane of a eukaryotic cell is selected from the group consisting of a ligand binding domain of a receptor protein, a ligand of a receptor, an antibody or an antigen binding fragment of an antibody, and a nanobody.

22. The vesicle according to any one of claims 1 to 21, wherein the polypeptide capable of binding to a membrane protein of a mammalian cell is a nanobody.

23. The vesicle according to any of claims 1 to 22, wherein the protein on the membrane is syndecan or glypican.

24. The vesicle according to any one of claims 1 to 23, wherein the therapeutic polypeptide is selected from the group consisting of a transcription factor, a small GTPase regulator protein, a regulator of a kinase, a regulator of a phosphatase, a regulator of a lipase, a kinase, a phosphatase and a lipase.

25. The vesicle according to any one of claims 1 to 23, wherein the therapeutic polypeptide is a complex of a Cas9 protein and a guide RNA.

26. The vesicle according to claim 25, wherein the guide RNA targets the mutation of an oncogene, such as RAS.

27. A kit of eukaryotic expression vectors comprising:
a first vector comprising a nucleotide sequence encoding a first fusion protein as defined in any one of claims 1-26, and
a second vector comprising a nucleotide sequence encoding a second fusion protein as defined in any one of claims 1-26.

28. The kit according to claim 27, wherein in the first fusion protein the polypeptide capable of binding to a membrane protein of a mammalian cell is a nanobody, and wherein in the second fusion protein the therapeutic polypeptide is a complex of a Cas9 protein and a guide RNA.

29. A eukaryotic cell comprising the vectors of the kit according to claim 27 or 28.

30. The eukaryotic cell according to claim 29, which is stably transfected with the vectors of the kit of claim 27 or 28.

31. The vesicle according to any of claims 1 to 26, or the kit according to claim 27 or 28, or the cell according to claim 29 or 30, or the secreted medium, or partially purified medium of said cells, comprising extracellular vesicles, for use as a medicament.


SEQUENCE LISTING (17 sequences)

SEQ ID NO: 1 - protein, 310 aa, Homo sapiens
MRRAALWLWLCALALSLQLALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 2 - protein, 17 aa, Homo sapiens
MRRAALWLWLCALALSL

SEQ ID NO: 3 - protein, 234 aa, Homo sapiens
QLALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKE

SEQ ID NO: 4 - protein, 25 aa, Homo sapiens
VLGGVIAGGLVGLIFAVCLVGFMLY

SEQ ID NO: 5 - protein, 34 aa, Homo sapiens
RMKKKDEGSYSLEEPKQANGGAYQKPTKQEEFYA

SEQ ID NO: 6 - protein, 298 aa, Homo sapiens
MSLYPSLEDLKVDKVIQAQTAFSANPANPAILSEASAPIPHDGNLYPRLYPELSQYMGLSLNEEEIRASVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 7 - protein, 110 aa, Homo sapiens
MSLYPSLEDLKVDKVIQAQTAFSANPANPAILSEASAPIPHDGNLYPRLYPELSQYMGLSLNEEEIRASVAVVSGAPLQGQLVARPSSINYMVAPVTGNDVGIRRAEIKQ

SEQ ID NO: 8 - protein, 162 aa, Homo sapiens
GIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPA

SEQ ID NO: 9 - protein, 88 aa, Homo sapiens
GIREVILCKDQDGKIGLRLKSIDNGIFVQLVQANSPASLVGLRFGDQVLQINGENCAGWSSDKAHKVLKQAFGEKITMTIRDRPFERT

SEQ ID NO: 10 - protein, 78 aa, Homo sapiens
FERTITMHKDSTGHVGFIFKNGKITSIVKDSSAARNGLLTEHNICEINGQNVIGLKDSQIADILSTSGTVVTITIMPA

SEQ ID NO: 11 - protein, 26 aa, Homo sapiens
FIFEHIIKRMAPSIMKSLMDHTIPEV

SEQ ID NO: 12 - protein, 22 aa, Homo sapiens
  REGION 1..22: note = signal peptide syndecan
MRRAALWLWLCALALSLQPALP

SEQ ID NO: 13 - protein, 10 aa, synthetic construct
  REGION 1..10: note = Myc epitope
EQKLISEEDL

SEQ ID NO: 14 - protein, 15 aa, synthetic construct
  REGION 1..15: note = Juxta CD4
  VARIANT 10: note = P, R
VKVLPTWSTXVQPMA

SEQ ID NO: 15 - protein, 59 aa, synthetic construct
  REGION 1..59: note = fragment
VLGGVIAGGLVGLIFAVCLVGFMLYRMKKKDEGSYSLEEPKQANGGAYQKPTKQEEFYA

SEQ ID NO: 16 - protein, 32 aa, synthetic construct
  REGION 1..32: note = fusion syndecan signal peptide - myc epitope
MRRAALWLWLCALALSLQPALPEQKLISEEDL

SEQ ID NO: 17 - protein, 74 aa, synthetic construct
  REGION 1..74: note = fusion CD4 juxta membrane - syndecan cytoplasmic domain
  VARIANT 10: note = R, P
VKVLPTWSTXVQPMAVLGGVIAGGLVGLIFAVCLVGFMLYRMKKKDEGSYSLEEPKQANGGAYQKPTKQEEFYA